(19) United States
(12) Patent Application Publication
(10) Pub. No.: US 2021/0145004 A1
(43) Pub. Date: May 20, 2021

(54) SURFACE, AIR, TEXTILE, PAINT, PLASTIC, SILICONE AND WOOD, POLYETHYLENE; METAL AND DERIVATIVES ANTIMICROBIAL PROPERTIES

(71) Applicant: UCAR HEALTH GMBH, Munich (DE)

(72) Inventor: Dilek UCAR, Cesme/Izmir (TR)

(21) Appl. No.: 17/046,976

(22) PCT Filed: Apr. 19, 2018
(86) PCT No.: PCT/TR2018/050176

§ 371 Date: Oct. 12, 2020

(51) Int. Cl. A01N 47/44; A61K 31/785

(52) U.S. Cl. CPC A01N 47/44; A61K 31/785

(57) ABSTRACT

This invention was related to elimination of pathogenic micro-organisms on various surfaces, and air especially hard surfaces where such organisms may stay active for relatively long periods of time, has long been a goal of those charged with cleaning and maintaining an antiseptic kitchens and bathrooms in the home, as well as in commercial and institutional settings such as hospitals, medical clinics, hotels and restaurants. A further goal has been to prevent the formation of allergens caused by growth of mold and mildew on bathroom surfaces. This invention further relates to cleaning, sanitizing, disinfecting and mold and mildew inhibiting compositions for non-porous hard surfaces such as glass (e.g., mirrors and shower doors), glazed porcelain, metallic (e.g. chrome, stainless steel, and aluminum), ceramic tile, enamel, fiberglass, Formica®, Corian® and plastic agent. This synthesis product having a pH of between 5.5 and 7.5 containing at least 3000 mg/l, preferably at least 2000 mg/l of a combination food protecting agent and guanidinium derivatives, particularly to combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts.

In accordance with the invention, a method for preventing, inhibiting or eliminating the growth, dissemination and/or accumulation of microorganisms on a susceptible surface (including but not limited to the formation of biofilms) comprises the step of contacting such surface with an antimicrobial agent, or composition thereof of the invention, with an amount sufficient to prevent, inhibit or eliminate such growth, dissemination and/or accumulation, i.e., with an effective amount.

As used herein “contacting” refers to any means for providing the compounds of the invention to a surface to be protected from, microbial growth and/or biofilm formation. Contacting can include spraying, wetting, immersing, dipping, painting, bonding, coating, adhering or otherwise providing a surface with a compound or composition in accordance with the invention. A “coating” refers to any temporary, semipermanent, or permanent layer, covering a surface. A coating can be a gas, vapor, liquid, paste, semi solid or solid. In addition a coating can be applied as a liquid and solidify into a hard coating. Examples of coatings include polishes, surface cleaners, caulks, adhesives, finishes, paints, waxes, polymerizable compositions (including phenolic resins, silicone polymers, chlorinated rubbers, coal tar and epoxy combinations, epoxy resins, polyamide resins vinyl resins, elastomers, acrylate polymers, fluoropolymers, polyesters and polyurethane, latex). Silicone resins, silicone polymers (e.g. RTV polymers) and silicone heat cured rubbers are suitable coatings for use in the invention and described in the art. Coatings can be ablative or dissolvable, so that the dissolution rate of the matrix controls the rate at which the compositions of the invention are delivered to the surface. Coatings can also be non-ablative, and rely on diffusion principals to deliver a composition of the invention to the target surface.

The compounds and compositions of the invention are also useful for preventing microbial growth and/or biofilms in industries outside of health-related industries, such as industrial systems wherein the presence of an aqueous environment leads to biofilm formation. Examples of such systems include metal working fluids, cooling waters (e.g. intake cooling water, effluent cooling water, recirculatin cooling water), and other recirculating water systems such as hose used in papermaking or textile manufacture.

Therefore, there is still a need to find and/or develop other bacterial, viral and fungicides for medium and surface disease control. Some of the chemical as such are already known. It is also known, that these compounds can be used as more healthy and applicable material.

### FIELD OF THE INVENTION

This invention relates to surface, air, textile, paint, plastic, silicone and wood, polyethylene and derivatives antimicrobial properties. The present invention relates to a combination polymeric guanidine derivative based on a diamine containing oxyalkylene chains between two amino groups, with the guanidine derivative representing a product of polycondensation between a guanidine acid addition salt and a diamine containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids and some natural products like phytotherapeutic plant extracts for the production of a liqid, powder and tablette form for antimicrobial activity.

The use of a polymeric guanidine derivative based on a diamine containing oxyalkylene chains between two amino groups, with the guanidine derivative representing a product of polycondensation between a guanidine acid addition salt and a diamine containing polyoxyalkylene chains between two amino groups, for the production of a drug composition with antimicrobial activity.

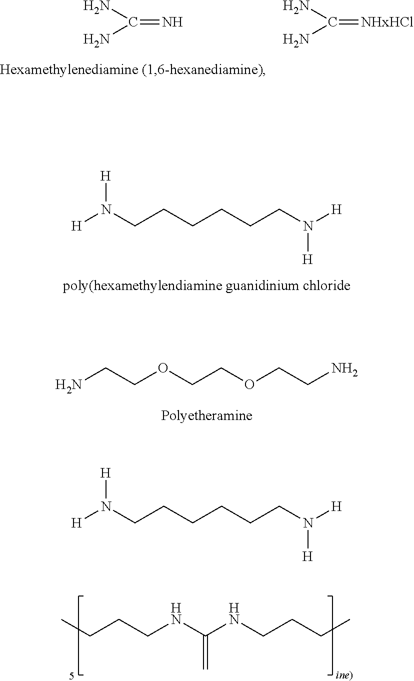

Sentez Material Chemical Structure

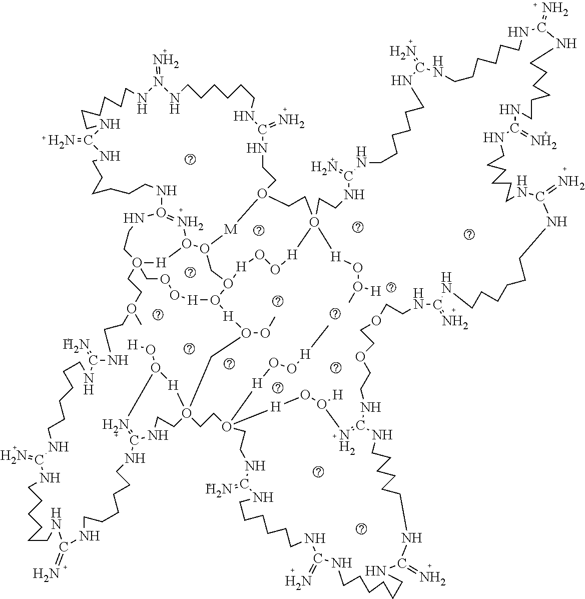

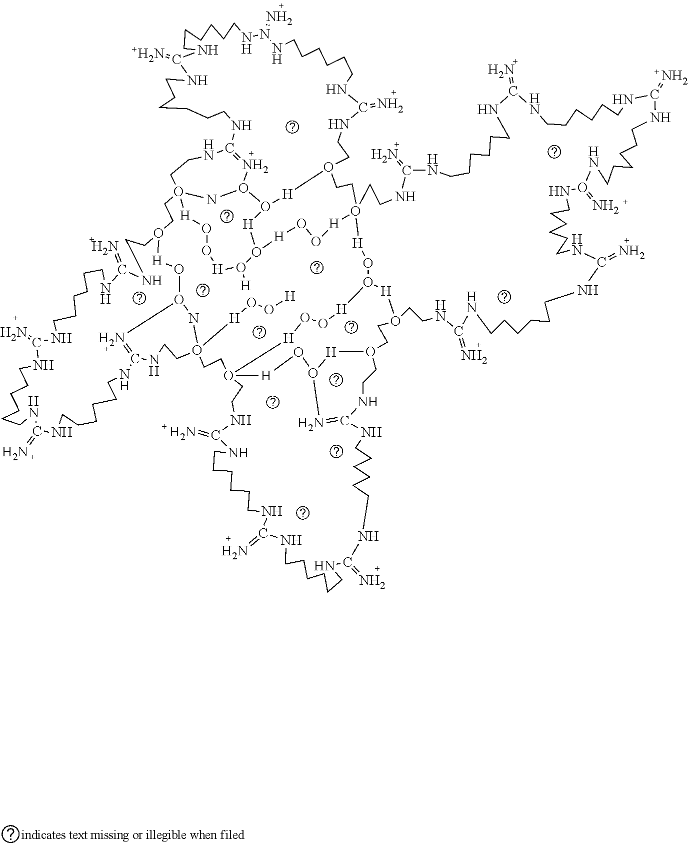

Synthesis Production of OSCAR

### BACKGROUND OF THE INVENTION

Elimination of pathogenic micro-organisms on various surfaces, especially hard surfaces where such organisms may stay active for relatively long periods of time, has long been a goal of those charged with cleaning and maintaining an antiseptic kitchens and bathrooms in the home, as well as in commercial and institutional settings such as hospitals, medical clinics, hotels and restaurants. A further goal has been to prevent the formation of allergens caused by growth of mold and mildew on bathroom surfaces. This invention further relates to cleaning, sanitizing, disinfecting and mold and mildew inhibiting compositions for non-porous hard surfaces such as glass (e.g., mirrors and shower doors), glazed porcelain, metallic (e.g. chrome, stainless steel, and aluminum), ceramic tile, enamel, fiberglass, Formica®, Corian® and plastic

In addition to their use in decontamination of land and equipment, the formulations also find use in household detergents for general disinfectant purposes. Moreover, some embodiments of the present invention can be used to prevent contamination of food with bacteria or fungi (e.g., non-toxic compositions). This can be done either in the food preparation process, or by addition to the food as an additive, disinfectant, or preservative.

The inventive compositions can be used on hard surfaces in liquid or aerosol form. Accordingly, the foregoing components are admixed with one or more suitable aqueous or non-aqueous carrier liquids. The choice of carrier is not critical. However, it should be safe and it should be chemically compatible with the inventive compositions. In some embodiments, the carrier liquid may comprise solvents commonly used in hard surface cleaning compositions. Such solvents should be compatible with the inventive compositions and should be chemically stable at the pH of the present compositions. Solvents for use in hard surface cleaners are described, for example, in U.S. Pat. No. 5,108,660, herein incorporated by reference in its entirety.

The present invention further relates to decontaminating a sample by treating the sample with the instant antimcrobial compositions such that bacteria, vims, fungi or spores on the surface are killed or disabled. The surfaces contemplated may be solid surfaces such as the surfaces in homes or industrial facilities or medical facilities or the surfaces of medical devices. Additionally the surface may be the surface of an organism and can be an internal or external organism surface. The surface further can be the surface of a food product.

The present compositions can be sprayed into an atmosphere to inactivate harmful microorganisms in the atmosphere. Such spray disinfectants are readily formulated by the skilled artisan and the choice of carrier is within the skill in the art.

Microbial contamination and biofilms adversely affect the health care industry and other industries wherein microbial contamination poses a health risk to humans such as public water supplies, and food production facilities. Infections involving implanted medical devices, for example, generally involve biofilms, where a sessile community provides a reservoir for an invasive infection. Antibodies and host immune defenses are ineffective in killing the organisms contained in a biofilm even though these organisms have elicited the antibody and related immune response. Antibiotics typically treat the infection caused by the planktonic organisms, but fail to kill those sessile organisms protected in the biofilm. Therefore, even if the contaminated medical device were removed from the host, any replacement device will be particularly susceptible to contamination from the residual microorganisms in the area from which the medical device was removed

Since the difficulties associated with eliminating biofilm-based infections and contamination are well-recognized, a number of technologies have developed to prevent or impair biofilm formation. These technologies include the development of various biocidal agents that are brought in contact with the contaminated or susceptible surface. However, any agent used to impair biofilm formation must be safe for use by humans and other non-target organisms. Biocides known to be effective at eliminating growth of unwanted microorganisms are generally toxic or otherwise harmful to humans, animals or other non-target organisms. Biocides known to be safe to non-target organisms, are generally less effective at preventing or eliminating microorganism growth, and require frequent application to the target surface.

Thus there is a need for antimicrobials that are safe, non-toxic, long-lasting and effective at controlling contamination and infection by unwanted microbial organisms, with minimal development of resistant or polyresistant microorganisms.

In one aspect, the present invention relates to novel, antimicrobial polymers. In another aspect, the present invention relates to antimicrobial pharmaceutical compositions and methods for treatment of microbial infections in a mammal.

In another aspect, the present invention relates to antimicrobial pharmaceutical compositions and methods for wound management.

In another aspect, the present invention relates to antimi crobial pharmaceutical compositions and methods for treatment of infections of the skin, oral mucosa and gastrointestinal tract.

In yet another aspect, the present invention relates to antimicrobial compositions and methods of preventing, inhibiting, or eliminating the growth, dissemination and accumulation of microorganisms on susceptible surfaces, particularly in a health-related environment.

The ionene polymers and compositions of the invention are also particularly useful for inhibiting the growth and dissemination, of microorganisms, particularly on surfaces wherein such growth is undesirable. The term “inhibiting the growth of microorganisms” means that the growth, dissemination, accumulation, and/or the attachment, e.g. to a susceptible surface, of one or more microorganisms is impaired, retarded, eliminated or prevented. In a preferred embodiment, the antimicrobial compositions of the inventtions are used in methods for inhibiting the growth of an organism on susceptible surfaces in health-related environments. The term “health-related environment” as used herein includes all those environments where activities are carried out directly or indirectly, that are implicated in the restoration or maintenance of human health. A health-related environment can be a medical environment, where activities are carried out to restore human health. An operating room, a doctor's office, a hospital room, and a factory making medical equipment are all examples of health-related environments. Other health-related environments can include industrial or residential sites where activities pertaining to human health are carried out such as activities including food processing, water purification, recreational water maintenance, and sanitation.

The term “susceptible surface” as used herein refers to any surface whether in an industrial or medical setting, that provides an interface between an object and the fluid. A surface, as understood herein further provides a plane whose mechanical structure, without further treatment, is compatible with the adherence of microorganisms. Microbial growth and/or biofilm formation with health implications can involve those surfaces in all health-related environments. Such surfaces include, but are not limited to, scalpels, needles, scissors and other devices used in invasive surgical, therapeutic or diagnostic procedures; implantable medical devices, including artificial blood vessels, catheters and other devices for the removal or delivery of fluids to patients, artificial hearts, artificial kidneys, orthopedic pins, plates and implants; catheters and other tubes (including urological and biliary tubes, endotracheal tubes, peripherally insertable central venous catheters, dialysis catheters, long term tunneled central venous catheters, peripheral venous catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses, (including breast implants, penile prostheses, vascular grafting prostheses, heart valves, artificial joints, artificial larynxes, otological implants), vascular catheter ports, wound drain tubes, hydrocephalus shunts, pacemakers and implantable defibrillators, and the like.

Other surfaces include the inner and outer surfaces of pieces of medical equipment, medical gear worn or carried by personnel in the health care settings and protective clothing for biohazard or biological warfare applications. Such surfaces can include counter tops and fixtures in areas used for medical procedures or for preparing medical apparatus, tubes and canisters used in respiratory treatments, including the administration of oxygen, solubilized drugs in nebulizers, and anesthetic agents. Additional surfaces include those surfaces intended as biological barriers to infectious organisms such as gloves, aprons and faceshields.

Surfaces in contact with liquids are particularly prone to microbial growth and/or biofilm formation. As an example, those reservoirs and tubes used for delivering humidified oxygen to patients can bear biofilms inhabited by infectious agents. Dental unit waterlines similarly can bear biofilms on their surfaces, providing a reservoir for continuing contami nation of the system of flowing and aerosolized water used in dentistry.

Other surfaces related to health include the inner and outer surfaces of equipment used in water purification, water storage and water delivery, and those articles involved in food processing equipment for home use, materials for infant care and toilet bowls.

In accordance with the invention, a method for preventing, inhibiting or eliminating the growth, dissemination and/or accumulation of microorganisms on a susceptible surface (including but not limited to the formation of biofilms) comprises the step of contacting such surface with an antimicrobial agent, or composition thereof of the invention, with an amount sufficient to prevent, inhibit or eliminate such growth, dissemination and/or accumulation, i.e., with an effective amount.

As used herein “contacting” refers to any means for providing the compounds of the invention to a surface to be protected from, microbial growth and/or biofilm formation. Contacting can include spraying, wetting, immersing, dipping, painting, bonding, coating, adhering or otherwise providing a surface with a compound or composition in accordance with the invention. A “coating” refers to any temporary, semipermanent, or permanent layer, covering a surface. A coating can be a gas, vapor, liquid, paste, semi solid or solid. In addition a coating can be applied as a liquid and solidify into a hard coating. Examples of coatings include polishes, surface cleaners, caulks, adhesives, finishes, paints, waxes, polymerizable compositions (including phenolic resins, silicone polymers, chlorinated rubbers, coal tar and epoxy combinations, epoxy resins, polyamide resins vinyl resins, elastomers, acrylate polymers, fluoropolymers, polyesters and polyurethane, latex). Silicone resins, silicone polymers (e.g. RTV polymers) and silicone heat cured rubbers are suitable coatings for use in the invention and described in the art. Coatings can be ablative or dissolvable, so that the dissolution rate of the matrix controls the rate at which the compositions of the invention are delivered to the surface. Coatings can also be non-ablative, and rely on diffusion principals to deliver a composition of the invention to the target surface. Non-ablative coatings can be porous or non-porous. A coating containing an antimicrobial agent of the invention freely dispersed in a polymer binder is referred to as a “monolithic” coating. Elasticity can be engineered into coatings to accommodate pliability, e.g. swelling or shrinkage of the surface to be coated.

Other means for contacting include a sustained or controlled release system that provides constant or prolonged release of an agent of the invention from a susceptible surface. This can be accomplished through the use of diffusional systems, including reservoir devices in which a core of an agent of the invention is surrounded by a porous membrane or layer, and also matrix devices in which the compound is distributed throughout an inert matrix. Materials which may be used to form reservoirs or matrices include silicones, acrylates, methacrylates, vinyl compounds such as polyvinyl chloride, olefins such as polyethylene or polypropylene, fluoropolymers such as polytetrafluorethylene or polypropylene, fluoropolymers such as polytetrafluorethylene, and polyesters such as terephthalates. Alternatively, the compositions of the invention may be mixed with a resin, e.g., polyvinyl chloride and then molded into a formed article, which integrally incorporates the compound to form a structure having a porous matrix which allows diffusion of the compound or a functional portion thereof into the surrounding environment. Microencapsulation techniques can also be used to maintain a sustained focal release of a compound of the invention.

Other means for providing the antimicrobial agents of the invention to a susceptible surface will be apparent to those of skill in the art.

The compounds and compositions of the invention are also useful for preventing microbial growth and/or biofilms in industries outside of health-related industries, such as industrial systems wherein the presence of an aqueous environment leads to biofilm formation. Examples of such systems include metal working fluids, cooling waters (e.g. intake cooling water, effluent cooling water, recirculating cooling water), and other recirculating water systems such as those used in papermaking or textile manufacture. Marine industries are also plagued by unwanted biofilms such as those that form on boat hulls and other marine structures.

Another embodiment of the present invention is an article comprising a polymer of the present invention in an amount sufficient to prevent, inhibit or eliminate the growth or dissemination of a microorganism or the formation of a biofilm, i.e., an “effective amount.” The polymer can be in the article or on the surface of the article. Preferably, the article is coated with a composition comprising an effective amount of a polymer of the present invention. Articles that are advantageously coated with a polymer of the present invention are those in which inhibition of the growth of microorganisms and/or biofilms is desirable, e.g., medical devices, medical furniture and devices exposed to aqueous environments. Examples of such articles are described above polymers) and silicone heat cured rubbers are suitable coatings for use in the invention and described in the art. Coatings can be ablative or dissolvable, so that the dissolution rate of the matrix controls the rate at which the compositions of the invention are delivered to the surface. Coatings can also be non-ablative, and rely on diffusion principals to deliver a composition of the invention to the target surface. Non-ablative coatings can be porous or non-porous. A coating containing an antimicrobial agent of the invention freely dispersed in a polymer binder is referred to as a “monolithic” coating. Elasticity can be engineered into coatings to accommodate pliability, e.g. swelling or shrinkage of the surface to be coated.

Other means for contacting include a sustained or controlled release system that provides constant or prolonged release of an agent of the invention from a susceptible surface. This can be accomplished through the use of diffusional systems, including reservoir devices in which a core of an agent of the invention is surrounded by a porous membrane or layer, and also matrix devices in which the compound is distributed throughout an inert matrix. Materials which may be used to form reservoirs or matrices include silicones, acrylates, methacrylates, vinyl compounds such as polyvinyl chloride, olefins such as polyethylene or polypropylene, fluoropolymers such as polytetrafluorethylene or polypropylene, fluoropolymers such as polytetrafluorethylene, and polyesters such as terephtha lates. Alternatively, the compositions of the invention may be mixed with a resin, e.g., polyvinyl chloride and then molded into a formed article, which integrally incorporates the compound to form a structure having a porous matrix which allows diffusion of the compound or a functional portion thereof into the surrounding environment. Microencapsulation techniques can also be used to maintain a sustained focal release of a compound of the invention.

Other means for providing the antimicrobial agents of the invention to a susceptible surface will be apparent to those of skill in the art.

The compounds and compositions of the invention are also useful for preventing microbial growth and/or biofilms in industries outside of health-related industries, such as industrial systems wherein the presence of an aqueous environment leads to biofilm formation. Examples of such systems include metal working fluids, cooling waters (e.g. intake cooling water, effluent cooling water, recirculatin cooling water), and other recirculating water systems such as hose used in papermaking or textile manufacture. Marine industries are also plagued by unwanted biofilms such as those that form on boat hulls and other marine structures.

Another embodiment of the present invention is an article comprising a polymer of the present invention in an amount sufficient to prevent, inhibit or eliminate the growth or dissemination of a microorganism or the formation of a biofilm, i.e., an “effective amount” The polymer can be in the article or on the surface of the article. Preferably, the article is coated with a composition comprising an effective amount of a polymer of the present invention. Articles that are advantageously coated with a polymer of the present invention are those in which inhibition of the growth of microorganisms and/or biofilms is desirable, e.g., medical devices, medical furniture and devices exposed to aqueous environments. Examples of such articles are described above.

Time Course of Antimicrobial Killing:

The purpose of this assay is to determine how rapidly biocidal compounds of the invention kill microorganisms.

One ml of Mueller Hinton broth with cations was inoculated with pathogen microorganism such as Bordetella pertussis, Borrelia burgdorferi, Brucella abortus, Brucella canis, Brucella melitensis, Brucella suis, Campylobacter jejuni, Chlamydia pneumonia, Chlamydia psittaci, Chlamydia trachomatis, Clostridium botulinum Clostridium difficile, Clostridium perfringens, Clostridium tetani, Corynebacterium diphtheria, Enterococcus faecalis, Enterococcus faecium, Escherichia coli (E. coli), Enterotoxigenic Escherichia coli (ETEC), Enteropathogenic E. coli, Erancisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumonia, Neisseria gonorrhoeae, Neisseria meningitidis, Pseudomonas aeruginosa, Rickettsia rickettsii, Salmonella typhi, Salmonella typhimurium, Shigella sonnei, Staphylococcus epidermidis, Staphylococcus saprophyticus, Streptococcus agalactiae, Streptococcus mutans Streptococcus pneumonia, Streptococcus pyogenes, Treponema pallidum, Vibrio cholera, Vibrio harveyi and Yersinia pestis An amount of synthesis producted equal to 5× the MIC was added at time point zero (Tq), and the mixture was incubated at 37° C. At selected time points (starting at Tq) samples were removed, serial ten-fold dilutions were prepared, aliquots were plated on Tryptic Soy Agar plates and were incubated overnight at 37° C. Colonies were then enumerated. For the antimicrobial polymers studied, killing occurs within 10 min at 5× the MIC.

In Vitro Toxicity:

Polymers with high antimicrobial activity were tested for in vitro toxicity against tissue culture cells. Cells were exposed to polymer for 18-24 hr, then were tested for metabolic activity using the mitochondrial redox indicator dye AlomarBlue® (AccuMed International, Inc., Chicago, Ill.) following the manufacturer's instructions.

Three different cell lines were used: AGS cells (an immor talized gastric cell line), CHO (Chinese Hamster Ovary) cells, and MDBK (Madin Darby Bovine Kidney) cells. Cells were plated into 96-well microtiter plates in RPMI or MEM culture medium containing 10% fetal bovine serum (FBS) at 1-5×10′*cells/well, and were grown 1-2 days 37° C. until confluent. Serial 2-fold dilutions of test synthesis product were prepared in MEM with 10% FBS. The medium was aspirated from confluent tissue culture cells, was replaced with 100 fil of polymer solution, and plates were incubated overnight at 37° C. The next day, cells were washed 2× with MEM (without phenol red or FBS), were overlaid with MEM lacking phenol red or FBS but containing AlomarBlue®, were incubated 4 hr at 37° C., and plates were read in a fluorimeter using 530 nm excitation and 590 nm for reading fluorescence. Values are expressed as percent of untreated controls, and the ED₅₀ was determined by regression analysis.

For the antimicrobial synthesis of the invention, the ED₅₀ was between 50-100× the MIC (data not shown).

In Vivo Toxicity:

In vivo toxicity of the polymer of Formula II was assayed in mice. Groups of 5 animals were dosed twice daily for 5 days by oral gavage at a dose of 10,100, or 500 mg/kg body weight. Animals were assessed daily, and deaths were recorded. The dose at which % the animals died was considered the LD₅₀. The LD50 was >90 mg/kg (Data not shown).

It is well known to add antimicrobial compounds to coatings (see for example US published applications 2011/0077363 and US 2010/0204357) and to polymeric matrices. Additionally, it is known to add antimicrobial compounds to coatings for medical devices and medical textiles such as in co-pending U.S. application Ser. Nos. 13/527,972 and 13/528,289 Additionally U.S. Pat. Nos. 7,520,897, 6,585,989 teach combinations of antimicrobials such as silver and tri-closan in polymeric matrices which give unexpected effects. [0004] Furthermore, the literature suggests combinations of certain antimicrobial polymers in combination with silver (ie. Langmuir, 2006, 22, 9820-9823, Biomacromolecules, 2008, 9, 2677-2681, Macromol. Rapid Commun. 2009, 30, 1350-1355 and ACS Applied Materials and Interfaces, 2012, 4, 460-465).

But until now, the additives that were appropriate for use in polymeric coating compositions or polymeric molding compositions, for example durable touch surfaces, were either slow-acting (e.g. silver ions), effective only against one or two bacteria, or had low effectiveness (<log 5 reduction) in quick-kill tests at practical concentrations.

In some embodiments, the antimicrobial agent of the invention is an antimicrobial preservative, attesting to the ability of the formulations of the invention to suppress microbial growth, reduce microbial infestation, treat products or surfaces to improve product resistance to microbial infestation, reduce biofilm, prevent conversion of bacteria to biofilm, prevent or inhibit microbial infection, prevent spoilage, retard or minimize or prevent quorum sensing, and retard microbial reproduction.

The antimicrobial agent is capable of endowing a product, or the product surface, with a biological resistance to at least one biological effect, which in the absence of such agent would eventually bring about a short-term or long-term damage to the product. In the context of the invention, the antimicrobial agent improves the product's resistance to a certain environmental condition. In some embodiments, the resistance to such a condition is resistance to biofouling.

In some embodiments, the microorganism is a bacteria, being selected, in some embodiments from Bordetella pertussis, Borrelia burgdorferi, Brucella abortus, Brucella canis, Brucella melitensis, Brucella suis, Campylobacter jejuni, Chlamydia pneumonia, Chlamydia psittaci, Chlamydia trachomatis, Clostridium botulinum Clostridium difficile, Clostridium perfringens, Clostridium tetani, Corynebacterium diphtheria, Enterococcus faecalis, Enterococcus faecium, Escherichia coli (E. coli), Enterotoxigenic Escherichia coli (ETEC), Enteropathogenic E. coli, Erancisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumonia, Neisseria gonorrhoeae, Neisseria meningitidis, Pseudomonas aeruginosa, Rickettsia rickettsii, Salmonella typhi, Salmonella typhimurium, Shigella sonnei, Staphylococcus epidermidis, Staphylococcus saprophyticus, Streptococcus agalactiae, Streptococcus mutans Streptococcus pneumonia, Streptococcus pyogenes, Treponema pallidum, Vibrio cholera, Vibrio harveyi and Yersinia pestis.

In other embodiments, the microorganism is a fungus, selected in some embodiments from Absidia corymbifera, Ajellomyces capsulatus, Ajellomyces dermatitidis, Arthroderma benhamiae, Arthroderma fulvum, Arthroderma gypseum, Arthroderma incurvatum, Arthroderma otae, Arthroderma vanbreuseghemii, Aspergillus flavus, Aspergillus fumigates, Aspergillus niger, Blastomyces dermatitidis, Candida albicans, Candida albicans var. stellatoidea, Candida dublinensis, Candida glabrata, Candida guilliermondii, Candida krusei, Candida parapsilosis, Candida pelliculosa, Candida tropicalis, Cladophialophora carrionii, Coccidioides immitis, Cryptococcus neoformans, Cunninghamella sp., Epidermophyton floccosum, Exophiala dermatitidis, Eilobasidiella neoformans, Eonsecaea pedrosoi, Geotrichum candidum, Histoplasma capsulatum, Hortaea werneckii, Issatschenkia orientalis, Madurella grisae, Malassezia furfur, Malassezia furfur complex, Malassezia globosa, Malassezia obtuse, Malassezia pachydermatis, Malassezia restricta, Malassezia slooffiae, Malassezia sympodialis, Microsporum canis, Microsporum fulvum, Microsporum gypseum, Microsporum gypseum complex, Microsporum gypseum, Mucor circinelloides, Nectria haematococca, Paecilomyces variotii, Paracoccidioides brasiliensis, Penicillium marneffei, Phialophora verrucosa, Pichia anomala, Pichia guilliermondii, Pneumocystis jirovecii, Pseudallescheria boydii, Rhizopus oryzae, Rodotorula rubra, Saccharomyces cerevisiae, Scedosporium apiospermum, Schizophyllum commune, Sporothrix schenckii, Stachybotrys chartarum, Trichophyton mentagrophytes, Trichophyton mentagrophytes complex, Trichophyton mentagrophytes, Trichophyton mentagrophytes, Trichophyton rubrum, Trichophyton tonsurans.

As used herein, the term “dry fogging”, or any lingual variation thereof, refers to the bringing together of the liquid to be treated (e.g., water) and the antimicrobial agent embedded or air diffusion and surface coated onto at least a part of the product's surface in such a way to allow elimination of organisms within the air and contaminated surface. Yet a further aspect provides a method for preventing growth of bacteria on a surface being in contact with dry fogging or dricetly sprayed the surface for at least a period of time, the method comprising incorporating the antimicrobial agent of the invention onto said surface. In some embodiments, the product's surface is surface coated with the antimicrobial agent as described herein.

The antimicrobial agents are present material impart the antimicrobial properties to the substrate. The surface may be associated with the surface in any way, e.g., Vander Waals forces, ionic bonding, hydrogen bonding, or through a coating linker such as a glue, forming stable coatings that exhibits minimal or no degradation or leaching, e.g., when exposed to an aqueous medium. As such, the dry fogging comprising the antimicrobial agents in accordance with the invention are safe for use in a variety of monthly applications.

The dry fogging substrates according to the invention may be used for (a) reducing or preventing bacterial infection without the need to use drug materials e.g., antibiotics, to the end product, the substrates may be any region of a storage container or a delivery system for use in food packaging, food and beverage containers, food and beverage preparation or disposing equipment, blood bags, proteins or pharmaceuticals. Alternatively, the antimicrobial agents of the invention may be used in the construction of a personal product or an industrial product such as devices used in sporting activities, orthodontic devices, face or breathing masks, pacifiers, contact lenses, adult products, food preparation surfaces, food packaging, surface, air, textile, paint, plastic, silicone and wood, polyethylene, metals and derivatives reusable water containers, hydration systems, water bottles, computer keyboards, telephones, rental car steering wheels, health club equipment, whirlpool spas and humidifiers to provide antimicrobial properties.

To evaluate the bactericidal efficiency of combination polymeric guanidine derivative based on a diamine containing oxyalkylene chains between two amino groups, with the guanidine derivative representing a product of polycondensation between a guanidine acid addition salt and a diamine containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids and some natural products like phytotherapeutic plant extracts for the production of a liqid, powder and tablette form for antimicrobial activity bactericidal efficiency was assessed for

Antimicrobial Activity:

HPP plates with 0.5% and 1% w/w of synthesis material from obtained combination polymeric guanidine derivative based on a diamine containing oxyalkylene chains between two amino groups, with the guanidine derivative representing a product of polycondensation between a guanidine acid addition salt and a diamine containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids were obtained by injection of the mixture. The mixtures were tested for virucidal and bactericidal efficacy against the E. coli bacteria by immersing samples in bacterial suspensions for predetermined period of time. The microorganisms used to assess biological activity are given in Table 1.

TABLE 1
The microorganisms used to assess biological activity

| | | | | | Bacteria |
|---|---|---|---|---|---|
| | No. of E. coli bacteria on the | | | | stitched |
| | surface of the sample (CFU/surface) | | | | into the |
| | After 24 | | After 76 | | suspension |
| | hours | | hours | | After 76 |
| Sample | Sidel | Side 2 | Sidel | Side 2 | hours |
| Synthesis product | — | — | 90 | 40 | — |
| without | | | | | |
| Synthesis product | — | — | 39 | 20 | — |
| 1% in HPP | | | | | |
| HPP (reference) | 1 × 10\hat{ }4 | 1 × 10\hat{ }4 | 1 × 10\hat{ }3 | 1 × 103 | 2 × 10\hat{ }4 |

One of the well known disinfecting agents is hydrogen peroxide and preparations thereof A representative of this group is a disinfecting preparation containing hydrogen peroxide, magnesium laurylsulphate, glycerin, sodium oleate, the disodium salt of EDTA, sodium benzoate and water (RU2108810 C1, 1998). This agent is intended for decontaminating surfaces in houses, sanitary appliances, linen, medical goods and its efficacy is not sufficient. It is not toxic to humans or animals.

Broadly known are bactericidal compositions exhibiting an increased activity containing lanthionine and a chelating agent. The suitable chelating agents are for example ethylenediaminotetraacetic acid (EDTA), its salts and citrate. (U.S. Pat. Nos. 5,260,271; 5,334,582)

Also described are bactericide compositions, which include cetyltrimethylammonium chloride as an active compound (DE 4326866,1995; U.S. Pat. Nos. 5,206,016; 5,575,991).

The related composition to the present invention is a disinfecting preparation which contains an active compound—a peroxide compound, a surfactant, a chelating complex and a solvent (RU20614497). This composition is active only when used at positive temperatures of 18-25° C. The prolongation of the bacteria inactivation is varied in the interval of 5-30 minutes.

The publication describes that said method is capable of suppressing the growth of microorganisms associated with putrefaction or deterioration. The publication does not refer to any effect of the method on medium pathogenic bacteria. One test is described wherein said combination of maltose and glycine is tested against putrefaction of a beef extract medium by Bacillus bacteria. The publication is directed to food spoilage by lactic acid bacteria rather than to food poisoning by gram-negative bacterial pathogens but guanisium base product is much more better this application. Guanidium can be used against food spoilage by “heat resistant indigenous or natural flora which survive the usual cooking or heat treating operation” and further against food poisoning outbreaks by enterotoxigenic microorganisms such as Micrococcus pyogenes or more commonly referred to as Staphylococcus. JP 57-008747 discloses that the addition of guanidium to raw materials for dried noodles has an antibacterial effect on the general group of coliform bacilli, and does not disclose use of guannidium and guanidium derivatievs materila such as combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts as antibacterial agent in a refrigerated food. Stonsaovapak et al. (Food 30th year Vol. 4 October-December 2000; XP002315133) discloses that glycine affects growth of two E. coli strains in TSB nutrient broth. In addition, the survival of E. coli O157:H7 in three ready to eat foods held at 4° C. is measured, however, without the addition of glycine but guanidium and derivatives produc is most effective.

Another publication describes the use of guanidium against molds and yeast and coliform: International Food Information, prolonging the storage life of foods by non-traditional preservation methods”. Some of the article describes the effect of guanidium on prolonging the storage life of preserved products. Raw pork goulash was used as test product. All samples were processed by heat-treatment. The results show that addition of guanidium has an effect on the growth of moulds and yeasts that are present in raw pork that has subsequently been heat-treated and pasteurised. The part of the microorganisms involving coliform microorganisms and arerobic spore-forming microorganisms is not significantly influenced by the presence of guanidium.

The active compound combinations or compositions according to the invention can be used for animal, fish and checken as such or, depend ing on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide-coated seed, suspension concentrates, suspo emulsion concentrates, soluble concentrates, suspensions, wettable powders, soluble powders, dusts and granules, water-soluble granules or tablets, water-soluble powders for the treatment of seed, wettable powders, natural products and synthetic substances impregnated with active compound, and also micro encapsulations in polymeric substances and in coating materials for seed, seedling and harvested product and also ULV cold-fogging and warm-fogging formulations.

The active compound combinations according to the invention can be present in (commercial) formulations and in the use forms prepared from these formulations as a mixture with other (known) active compounds, such as insecticides, attractants, sterilants, bactericides, acaricides, nematicides, fungicides, growth regulators, herbicides, fertilizers, safeners and Semiochemicals.

The treatment according to the invention of the animal, fish and checken with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment a solution for seed treatment, a water-soluble powder for slurry treatment, by incrusting, by coating with one or more layers, etc. It is furthermore possible to apply the active compounds by the ultra-low volume method, or to inject the active compound preparation or the active compound itself into the soil.

The present invention is directed to the use of combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts as antibacterial agent against the gram-negative bacterial and fungie pathogens combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts in refrigerated animal, fish and checken foods or refrigerated drinks with the proviso that in addition to said guanidium and/or guanidium derivative no hetero-saccharide containing macromolecule is used, nor 1,5-D-anhydrofructose is used as antibacterial agent in said foods. While in some the prior art documents complicated guanidium compounds are mentioned which are stated to have antibacterial properties, the present invention is directed to the use of “simple” guanidium compounds such as containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheraniine derivatives (JEFFAMINE EDR-148), Polietheraniine (Triethyleneglycol diamine (TEGDA) enzymes, PGPR, amino acids, antioxidants like humic acids and some natural products like phytotherapeutic plant extracts. The medium present in real food and food products products comprises proteins and fats, has a specific mobility of the liquids present, adsorption or incorporation of the guanidium in the food product may occur. Without being committed to a theory, it is thought that the fact that guannidium is an amin group and a natural building block of food and food products and is abundantly present in food constituents, causes it to interfere in a rather unpredictable way in real food and food products.

We have found that combinations of containing containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids and some natural products like phytotherapeutic plant extracts can effectively be used as a sole antibacterial agent in concentrations that are still acceptable in food and food products without negatively affecting the product quality with respect to for example taste and texture. We have found that combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts can be used as sole antibacterial agent for preservation purposes and further to prevent the consequences of contamination of fruit, vehetables, animal, fish and checken food and drink products as food poisoning by pathogenic bacteria due to temperature-abuse and/or contamination using antimicrobial surface of touch surface such as polyethylene, metal, plastic. It is not needed to add an auxiliary antibacterial agent to achieve the desired preservation effect in contrast to the results described in above-mentioned patents. This results not only in lower material costs but also in a higher product quality. Products are obtained with less auxiliary ingredients added while maintaining and even improving the quality and shelf life of said products. Further, this is in line with legislation that is aimed at minimisation of the use of additives in food applications. Furthermore, the products obtained are also protected against the consequences of temperature-abuse or contamination.

Containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids are found to be very effective as antibacterial agent in meat applications including fish and poultry, both cured and uncured meat and fresh meat both direcly using and storage position with antimicrobial surface of touch surface food contant material such as polyethylene, metal, plastic. The gram-negative bacterial pathogens Escherichia Coli, Enterobacter sakazakii, Salmonella, pseudomonas, escheria coli, entereobacter aerogenes, coliform, legionalla, and Campylobacter and in particular against Salmonella typhimurium, Salmonella enteriditis, Escherichia Coli O157:H7 and Campylobacter jejuni are often found in these types of applications. Above-mentioned bacteria are relatively insensitive to control of pH, water activity or addition of nitrite. Acid, salt or nitrite would have to be added in high concentrations in order to achieve some effect on bacteria growth, but these high concentrations negatively affect the product quality in terms of a bad taste and a loss of texture of the meat. The use of combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts as antibacterial agent is found to be effective against said bacteria without loss of taste and without loss of texture both directly application and and/or contamination using antimicrobial surface of touch surface such as polyethylene, metal, plastic. Furthermore, the above-mentioned methods and alternative processing techniques as e.g. heat treatment for preservation do not prevent food poisoning as consequences of temperature-abuse and/or contamination. Examples of fresh and dry vegetable, fruit and fresh meat are beef, beef steak, beef oxtails, neckbones, short ribs, beef roasts, stew meat, beef briskets, pork, pork chops, por steaks, cutlets, pork roasts, lamb, veal, game goat, filet americain, steak tartar, sushi, or carpaccio, chicken, turkey, duck and other poultry directly and/or contamination using antimicrobial surface of touch surface such as polyethylene, metal, plastic. Some of these fresh meat applications are to be consumed raw, while others are consumed after application of only partial heat treatment, intentionally applied as e.g. for medium cooked steak or unintentionally applied due to improper preparation or improper handling of the food products. The use of containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids as antibacterial agent ensures food safety even in the case of partial heat-treatment. The antibacterial activity not only includes bacterio static activity preventing further bacterial growth but also includes for some bacteria bacteriocidal activity that actually reduces the bacterial number. containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids of 0.5 to 2 wt % based on total weight of product were found to be effective as antibacterial agent for escheria coli, entereobacter aerogenes, coliform, legionalla and glycine concentrations of 0.5 to 1.8 wt % based on total weight of product were found to be suited in ensuring taste of the product. Combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts concentrations of 0.25 to 2 wt % based on total weight of product were found to be effective as antibacterial agent for E. Sakazakii and containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids of 0.5 to 1.5 wt % based on total weight of product were found to be suited in ensuring taste of the product.

Combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts concentrations of 0.2 to 3 wt % based on total weight of product show antibacterial activity against Salmonella, and in particular Salmonella typhimurium and Salmonella enteriditis. Combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts concentrations of 0.2 to 1.5 wt % based on total weight of product were found to be suited in ensuring taste of the product. Tests showed that a concentration of about 1 to 1.8 wt % of combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts based on total weight of product starts to affect the taste of said product. In said product no auxiliary antibacterial agents and no other taste affecting ingredients were present. A combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts concentration above 1.5 wt % based on total weight of the product gives the product a sweet taste. Dependent on the type of product this sweet taste is acceptable or not. In sweet drinks for example the sweetening effect of guanidium is not considered a problem. Accordingly the maximally acceptable combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts concentration in terms of not negatively affecting taste can be increased to concentrations above 1.8 wt % guanidium based on total weight of the product. Further, dependent on the presence of other taste affecting ingredients in the product as for example masking agents, the maximum concentration of combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts can also be increased up to a point at which the taste starts to be negatively affected by the presence of gundium derivative. It was found that the use of guanidium and/or its derivatives according to the invention as antibacterial agent in refrigerated foods and refrigerated drinks may be combined with one or more combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts.

The related art of interest describes various food supplements, but none discloses the present invention. There is a need for a powdered food protecting composition which supplies a variety of nutrients and flavors for enriching and flavoring ones food, but also curbing ones appetite to prevent overeating. The related art will be discussed in the order of perceived relevance to the present invention.

U.S. Pat. No. 4,710,387 issued on Dec. 1, 1987, to Dirk J. D. Uiterwaal et al. describes a nutritional supplement preparation for pregnant and breast-feeding women based on milk constituents and the process of preparation. The composition contains by weight 10-20% protein, 16-28% fat, 43-65% carbohydrates, at most 3.5% moisture, minerals, trace elements, and vitamins such as calcium, phosphorus, magnesium, copper, zinc, iodine, iron, vitamins A, B1, B6, C, D3, E, niacin, folic acid, and optionally flavoring and/or colorant. The composition is distinguishable for being tailored to supply nutrients to a pregnant or breast-feeding woman and requires large amounts of fat (linoleic acid) and carbohydrates (lactose, dextrins and sucrose).

U.S. Pat. No. 5,770,217 issued on Jun. 23, 1998, to Frank J. Kutilek, III et al. describes a dietary pilled supplement comprising herbs, herbal extracts, vitamins, minerals, and amino acids effective in modulating hematological toxicities, enhancing the immune system and maintaining appetite and weight. The supplement contains a large amount of crucifer extract (8-12 wt. %) and ascorbic acid or vitamin C (8-13 wt. %). The amino acids include glutathione, L-cysteine and L-methionine. The composition is distinguishable for requiring crucifer extract and amino acids in pill form.

U.S. Pat. No. 4,348,379 issued on Sep. 7, 1982, to Horst Kowalsky et al. describes a dietetic composition for natural digestion regulation comprising in parts by weight each of 50-150 of whole fleawort seeds, whole linseed, wheat bran, and lactose. A binding agent based on natural rubber and optionally, flavor and/or food color. The composition is distinguishable for requiring fleawort seeds and whole linseed.

U.S. Pat. No. 4,440,760 issued on Apr. 3, 1984, to Rex E. Newnham describes a food supplement for the relief of arthritic conditions comprising in parts by weight of 2-500 of sodium tetraborate, 150 each of the dried herbs Gauaiacum, Berberis and Harpagophytum, 1 ppm Rhus-tox and/or Bryonia, gum arabic as binder, starch as a disintegration aid, and magnesium stearate as a lubricating aid in tablet forming. The composition is distinguishable for requiring sodium tetraborate and several dried herbs not required in the present invention.

U.S. Pat. No. 5,332,579 issued on Jul. 26, 1994, to Anthony J. Umbdenstock describes a nutritional supplement for optimizing cellular health of recovering drug addicts, alcoholics, smokers, etc., comprising: 1,500-15,000 I.U. vitamin A; 5,000-45,000 I.U. Beta-carotene; 33-300 mg. vitamin B1; 50-1,000 mg. vitamin B6; 30-300 mcg. vitamin B12; 20-500 mg. niacin; 100-2,000 mg. niacinamide; 100 mg. vitamin C; 5-100 mg. magnesium; 10-100 mg. zinc; 50-1,000 mg. valerian root; at least two minerals selected from the group consisting of calcium, 20-500 mcg. chromium, copper, iron, 5-1,000 mg. manganese, and selenium; and at least four additional vitamins, herbs, and amino acids selected from the group consisting of 100-1,000 I.U. vitamin D3, 10-800 I.U. vitamin E, 5-100 mg. vitamin B2, 100-1,000 mcg. biotin, 50-500 mg. pantothenic acid, 70-900 mg. choline, 100-1,000 mg. inositol, 50-1,000 mg. glutamic acid, 50-1,000 mg. glutamine, and echinachea. The composition is distinguishable for omitting beans, peas, berries, and grains. U.S. Pat. No. 5,656,312 issued on Aug. 12, 1997, and U.S. Pat. No. 5,834,048 issued on Nov. 10, 1998, to Udo Erasmus et al. describes a daily dietary food supplement composition packaged in a sealed pouch for humans comprising at least by weight proportions, 71-73% flax seeds, 5% yeast, 6% rice and bran yeast, 2% liver, 2% alfalfa, 1% bone, 2% carrot, 2% apple, 0.07% kelp. 0.01% lecithin, 0.01% garlic, 0.02% taurine, 0.01% equiteum herb, and 0.01% carnitine. The composition must be prepared at a temperature below 100° F. for less than 20 minutes and in the limited illumination of red light. The food composition is distinguishable for requiring liver, yeast, flax seeds, garlic, and taurine as well as limited heating and lighting conditions.

U.S. Pat. No. 5,925,377 issued on Jul. 20, 1999, to Teja D. Gerth et al. describes a dietary supplement composition combining amino acids, minerals, herbs, vitamins, diuretics, and digestive enzymes. For example, D,L-phenylalanine is combined with tyrosine, L-glutamine and St. John's wort to act as an appetite depressant while L-carnitine is combined with chromium picolinate to work as fat directors to convert stored body fat into energy. The composition is distinguishable for requiring diuretics and digestive enzymes.

U.S. Pat. No. 5,976,579 issued on Nov. 2, 1999, and U.S. Pat. No. 6,143,332 issued on Nov. 7, 2000, to Linsey McLean describes a nutritional supplement for the prevention and treatment of excessive intestinal permeability comprising at least 50 wt. % nutritional buffers (calcium carbonate), amino acid chelates (selenium, copper, zinc, manganese, iodine, and chromium), minerals, vitamins (A, B-complex, D, and E), antioxidants, free radical scavengers, and intestinal tract-soothing herbs. The composition is distinguishable for requiring buffers, chelates, antioxidants, and free radical scavengers.

U.S. Pat. No. 6,238,672 B1 issued on May 29, 2001, to Jau-Fei Chen describes dietary supplements containing dehydrated cactus fruit juice and ginseng berry juice for food products, drinks, capsules, and tablets. The supplement is distinguishable for requiring cactus fruit juice and ginseng berry juice.

U.S. Pat. No. 6,264,995 issued on Jul. 24, 2001, to Thomas Newmark et al. describes a herbal composition for reducing inflammation in bones and joints comprising holy basil, tumeric, ginger, green tea, rosemary, huzhang, Chinese goldthread, barberry, oregano, and scutellariae baicalensis. The composition is distinguishable for its medicinal characteristics.

U.S. Pat. No. 6,274,189 B1 issued on Aug. 14, 2001, to Massoud Kazemzadeh describes a reduced-flatulence, legume-based snack foodswhich comprises saccharides depleted legumes (field bean, white pea bean, Tepary bean, Mung bean, lima bean, Broad-bean, chickpea, lentil beans, peanuts, buckwheat, and flax) grain-based ingredients (cereal grain, oil seed, oil seed flour, and mixtures thereof), water, and processing aids (inorganic and organic compounds of calcium, potassium, sodium, and mixtures thereof) processed with a developed matrix having a high crunch texture and a low fat absorption when cooked. The compositions are distinguishable for requiring cooking.

German Patent Application No. DE 31 43 926 A1 published on May 11, 1983, for Kurt Jesselring et al. describes a dietetic composition containing bran and/or pectin, vitamins, minerals, customary auxiliaries and carriers, and an anti-thrombotically active fraction derived from Basidiomycetes such as Auricularia, Himeola auricula judae, Polyporus ovinus, Polyporus giganteus, and Sparassis crispa. The composition is distinguishable for requiring an antithrombotically active fraction.

German Patent Application No. DE 44 16 402 A1 published on Nov. 30, 1995, for Harro Carstens et al. describes an immunity improving food protecting composition comprising ethanolic extra extracts of medicinal herbs (aloe), vegetable oils containing eugenol, and, optionally, conventional stabilizers and additives. The medicinal herb extracts have a detoxifying effect through the stomach and intestinal tract. The composition is distinguishable for requiring only herb extracts, vegetable oils and eugenol.

French Patent Application No. 2 737 849 published on Feb. 21, 1997, for Jean P. Curtay et al. describes an orally administered food supplement for adults over forty years of age comprising: (1) an excipient (gum arabic or starch); (2) mineral salts (calcium carbonate, magnesium carbonate, zinc citrate; (3) vitamins B1, B2, B6, B8, B9, B12, C, E, and PP; (4) beta-carotene, (5) borage oil (herb); (6) fish oil; and (7) methionine. The composition is distinguishable for requiring beta-carotene, borage oil and methionine.

German Patent Application No. DE 196 53 100 A1 published on Jul. 23, 1998, for Adolph Metz describes a lactose-containing magnetic capsule food supplement comprising: (1) ferromagnetic magnetite; (2) piezoelectric rock crystal (silica); (3) magnesite powder (magnesium carbonate); (4) ginseng root, taiga root, mistletoe, ginkgo biloba leaves, hawthorn flowers or leaves, horse chestnut leaves, milk thistle, balm mint leaves, St. John's wort, speedwell, linden flowers, arnica flowers, lesser centaury (Erythraea centaurium), marigold flowers, yarrow (Achillea millefolium), red soapwort, and calamus root; and (5) vitamins A, C, E, aneurin, riboflavin, pyridoxine, B12, and Q10; reduced glutathione, glutamine, cysteine, methionine; (6) Ca-, Mg- and K-citrate; (7) E. Coli or Lactobacillus acidophilus; (8) heartwood of Thuaja plicata; (9) oak bark; (10) aspirin and/or willow bark; (11) zinc, selenium and manganese; and (12) lactose, starch and dextrose. The composition is distinguishable for requiring a vast variety of exotic herbs, vitamins, lactose, minerals, and a magnetic constituent.

W.I.P.O. Patent Application No. WO 98/00024 published on Jan. 8, 1998, and W.I.P.O. Patent Application No. WO 98/47376 published on Oct. 29, 1998, for Houn S. Hsia describes a diet supplement composition to increase the level of high density lipoprotein (HDL) and calcium ions, and to decrease the levels of free radicals and glucose in human blood plasma comprising; (1) anti-oxidants selenium, vitamins A, B, C, D, and E, and fruit or vegetable juice concentrates; (2) green barley composition; (3) tincture of ginkgo biloba extract; and (4) minerals. The composition is distinguishable for requiring minerals, ginkgo biloba, and fruit and vegetable juice concentrates.

German Patent Application No. DE 199 07 586 A1 published on Aug. 24, 2000, for Waldemar Braun et al. describes a daily nutritional composition comprising (a) a basic kit for constant circadian dosage combined with (b) an “add-on” supplement used in time-dependent amounts. The basic kit contains specific amounts of various vitamins and minerals including beta-carotene, vitamins B1, B2, B6, B12, niacin, pantothenic acid, biotin, folic acid, phylloquinone, calcium, magnesium, manganese, zinc, iron, selenium, chromium, molybdenum, copper, and iodine. The “add-on” composition contains apple vinegar powder, artichoke extract, carnitine, guarina, silica, creatine, lecithin, and taurine. The compositions are distinguishable for requiring minerals and the “add-on” composition.

None of the above inventions and patents, taken either singularly or in combination, is seen to describe the instant invention of flavoring and therapeutic effect. Thus, a food protecting composition composition solving the aforementioned problems is desired.

### SUMMARY OF THE INVENTION

The object of the present invention is to provide a highly effective universal disinfecting, antiseptic and bactericidal, fungicidal or virucidal composition, which is useful in a broad range of positive and negative temperatures and in increasing the term of microbiocidal and disinfectant action. A further objective of the invention is to enhance the length of time of the microbiocidal or disinfectant action. The present composition is suitable for a long-term storage, is safely used, and exhibits high bactericidal, virucidal, fungicidal, and sporocidal activity and is nontoxic to animals and humans. The present antimicrobial and anti-sporicidal compositions are useful in a wide variety of utility areas. These compositions are useful as topical applications in the treatment of microbiocidal infections in a subject. Applicants' compositions can be applied to various surfaces and when so applied these compositions serve as sterilizers or sanitizers. Similarly, the present compositions can be used in application areas such as, for example, in swimming pools, spas, etc.,as a laundry soap or detergent additive, as a paint or surface coating additive, as a natural or synthetic surface preservative such as the prevention of microfloral growth on surfaces such as polymers, plastics or wood, as a hard surface or carpet sanitizer. These compositions are generally useful in controlling and/or elimination of microflora and spores in many industrial, medical, agricultural, veterinary and domestic applications. Additionally, the present compositions can be employed to sterilize or disinfect gaseous environments including, for example, the cleansing of the atmosphere in homes and industrial sites, as well as airplanes, etc.

In accordance with these and other aspects, the present invention provides novel ionene polymers having antimicrobial activity. “combination polymeric guanidine derivative based on a diamine containing oxyalkylene chains between two amino groups, with the guanidine derivative representing a product of polycondensation between a guanidine acid addition salt and a diamine containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids and some natural products like phytotherapeutic plant extracts” or “synthesis product,” as used in the present invention, are cationic polymers or copolymers with amine groups in the main polymeric chain or backbone of the polymer, providing a positive charge. The synthesis of this invention have been found to be non-irritating and low in toxicity to warm-blooded animals. The present invention also provides antimicrobial compositions comprising synthesis product and methods for treating microbial infections in mammals comprising the step of administering to a mammal, a therapeutically effective amount of at least one antimicrobial composition of the invention. The present invention further provides antimicrobial compositions comprising at least one synthesis and methods for preventing, inhibiting or eliminating the growth, dissemination, and/or the accumulation of microorganisms on a susceptible surface such as surface, air, textile, paint, plastic, silicone and wood, polyethylene and derivatives.

### DETAILED DESCRIPTION OF THE INVENTION

The present invention relates to methods for the protection of poultry (checken, duck), fish and animal (cow, pig, deer, sheep) dairy product and derivatives such as meat, yogurt, butter, cheese, sausage, and chicken, fish and fish preserves against decay caused by certain processes, storage diseases or disorders expressed in storage conditions, which meat, checken and fish and derivatives have been treated before consum with combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts.

Food protecting agent composition having a pH of between 5.5 and 7.5 containing at least 2000 mg/l, preferably at least 1800 mg/l of a combination food protecting agent and guanidinium derivatives, particularly to combinations of oligo(2-(2-ethoxy)ethoxy ethyl guanidinium chloride), poly(hexamethylendiamine guanidinium chloride), polyetheramines, triethyleneglycol diamine, enzymes, PGPR, amino acids, antioxidants such as humic acids and some natural products like phytotherapeutic plant extracts.

The purpose of any raw and procesesing pruduct for catte, pig etc, fish and chicken is to prevent the development of diseases that might impair the final quality of the products. The system for catte, pig etc, fish and chicken application of food protection products on catte, pig etc, fish and chicken during packing aims to safeguard the health of the catte, pig etc, fish and chicken during the period of storage and transport to the final consumer. Since, moreover, the environmental and economic requirements imposed on modern-day fungicides are continually increasing, with regard, for example, to the spectrum of activity, toxicity, selectivity, application rate, formation of residues, and favorable preparation ability, and since, furthermore, there may be problems, for example, with resistances developing to known active compounds, a constant task is to develop new fungicide agents which in some areas at least have advantages over their known counterparts. Therefore, there is still a need to fmd and/or develop other bacterial, viral and fungicides for storage disease control. Some of the chemical as such are already known. It is also known, that these compounds can be used as more helaty and appliceable material.

This invention relates to the use of an antibacterial agent against gram-negative bacterial pathogens in foods. Said antibacterial agent is in particular applied in refrigerated foods and more in particular in fresh or cooked meat (including poultry and fish) products. Further, said antibacterial agent is in particular used against bacteria and bacteria from the genus Fusarium spp., e.g. Fusarium semitectum, Fusarium moniliforme, Fusarium solani, Fusarium oxysporum; Verticillium spp., e.g. Verticillium theobromae; Nigrospora spp.; Botrytis spp., e.g. Botrytis cinerea; Geotrichum spp., e.g. Geotrichum candidum; Phomopsis spp., Phomopsis natalensis; Diplodia spp., e.g. Diplodia citri; Alternaria spp., e.g. Alternaria citri, Alternaria alternata; Phytophthora spp., e.g. Phytophthora citrophthora, Phytophthora fragariae, Phytophthora cactorum, Phytophthora parasitica; Septoria spp., e.g. Septoria depressa; Mucor spp., e.g. Mucor piriformis; Monilinia spp., e.g. Monilinia fructigena, Monilinia laxa; Venturia spp., e.g. Venturia inaequalis, Venturia pyrina; Rhizopus spp., e.g. Rhizopus stolonifer, Rhizopus oryzae; Glomerella spp., e.g. Glomerella cingulata; Sclerotinia spp., e.g. Sclerotinia fruiticola; Ceratocystis spp., e.g. Ceratocystis paradoxa; Penicillium spp., e.g. Penicillium funiculosum, Penicillium expansum, Penicillium digitatum, Penicillium italicum; Gloeosporium spp., e.g. Gloeosporium album, Gloeosporium perennans, Gloeosporium fructi genum, Gloeosporium singulata; Phlyctaena spp., e.g. Phlyctaena vagabunda; Cylindrocarpon spp., e.g. Cylin drocarpon mali; Stemphyllium spp., e.g. Stemphyllium vesicarium; Phacydiopycnis spp., e.g. Phacydiopycnis malirum; Thielaviopsis spp., e.g. Thielaviopsis paradoxy; Aspergillus spp., e.g. Aspergillus niger, Aspergillus carbonarius; Nectria spp., e.g. Nectria galligena; Pezicula spp., Escherichia Coli, Enterobacter sakazakii, Salmonella, pseudomonas, escheria coli, entereobacter aerogenes, coliform, legionalla, Salmomella spp., Campylobacter spp., Staphylococcus aureus, Listeria monocytogenes, Bacillus cereus, Clostridium spp., Escherichia coli O157:H7, Shigella spp., Yersinia enterocolitica, Vibrio spp., Brucella spp. ve Aeromonas spp. and Campylobacter in said food. Conventionally, bacterial, viral and fungui growth in food is controlled and/or prevented by means of pH regulation, water activity control, addition of quality preserving agents as e.g. nitrite and/or using various processing techniques as for example heat treatment, irradiation or high-pressure treatment. However, when controlling gramnegative bacterial pathogens the above-described measures are often either insufficient, undesirable or not suitable for the type of food. For instance, controlling the water activity in products is possible by means of e.g. salt addition. Controlling or preventing bacterial growth in products by means of salt addition however requires high salt concentrations. Said high concentrations often lead to a loss of taste because the product becomes too saltyand not applicable. Further, a too high salt dosage is also not desired with respect to health issues as for example heart and vascular diseases or blood pressure. Furthermore, in protein-containing products as for example meat (this is including fish and poultry) said high salt concentrations may lead to deterioration of the texture of the product. As gram-negative bacterial pathogens and some fungie such as Fusarium spp., e.g. Fusarium semitectum, Fusarium moniliforme, Fusarium solani, Fusarium oxysporum; Verticillium spp., e.g. Verticillium theobromae; Nigrospora spp.; Botrytis spp., e.g. Botrytis cinerea; Geotrichum spp., e.g. Geotrichum candidum; Phomopsis spp., Phomopsis natalensis; Diplodia spp., e.g. Diplodia citri; Alternaria spp., e.g. Alternaria citri, Alternaria alternata; Phytophthora spp., e.g. Phytophthora citrophthora, Phytophthora fragariae, Phytophthora cactorum, Phytophthora parasitica; Septoria spp., e.g. Septoria depressa; Mucor spp., e.g. Mucor piriformis; Monilinia spp., e.g. Monilinia fructigena, Monilinia laxa; Venturia spp., e.g. Venturia inaequalis, Venturia pyrina; Rhizopus spp., e.g. Rhizopus stolonifer, Rhizopus oryzae; Glomerella spp., e.g. Glomerella cingulata; Sclerotinia spp., e.g. Sclerotinia fruiticola; Ceratocystis spp., e.g. Ceratocystis paradoxa; Penicillium spp., e.g. Penicillium funiculosum, Penicillium expansum, Penicillium digitatum, Penicillium italicum; Gloeosporium spp., e.g. Gloeosporium album, Gloeosporium perennans, Gloeosporium fructi genum, Gloeosporium singulata; Phlyctaena spp., e.g. Phlyctaena vagabunda; Cylindrocarpon spp., e.g. Cylin drocarpon mali; Stemphyllium spp., e.g. Stemphyllium vesicarium; Phacydiopycnis spp., e.g. Phacydiopycnis malirum; Thielaviopsis spp., e.g. Thielaviopsis paradoxy; Aspergillus spp., e.g. Aspergillus niger, Aspergillus carbonarius; Nectria spp., e.g. Nectria galligena; Pezicula spp., Escherichia Coli, Enterobacter sakazakii, Salmonella, pseudomonas, escheria coli, entereobacter aerogenes, coliform, legionalla, Salmomella spp., Campylobacter spp., Staphylococcus aureus, Listeria monocytogenes, Bacillus cereus, Clostridium spp., Escherichia coli O157:H7, Shigella spp., Yersinia enterocolitica, Vibrio spp., Brucella spp. ve Aeromonas spp. and Campylobacter are notoriously present in protein-containing products such as milk, meat, cheese etc., controlling the water activity is often not a viable solution. Also pH regulation of the pH as means for controlling bacterial growth can cause loss of taste of the product and/or loss of texture of the product, especially in protein-rich food. Furthermore, some gram-negative pathogenic bacteria are relatively insensitive to acid addition. For instance growth of Campylobacter bacteria and Salmonella bacteria can be stopped at a pH lower than respectively 4.0 and 3.8, which pH is for some food applications undesirable because of its effect on taste, texture and not convenient appliceable methods.

Nitrite is added in cured meat (including poultry and fish) applications for the purpose of preserving product quality. Nitrite is able to stop bacterial growth of some types of bacteria as for example Clostridium. In some cases nitrite is added as colouring agent to maintain a certain colour in the meat product. Due to this colouring effect of nitrite it is not desirable for all meat applications. Examples of uncured, not nitrite-containing, product applications are (German) sausages, chicken and turkey meat and roast beef. As mentioned above, especially gram-negative bacterial pathogens are often present in these food products. At present legislation is aimed at minimisation of the use of nitrite in food applications. It goes without saying that processing techniques as for example heat treatment, irradiation or high-pressure treatment as method for preservation of products is not always applicable to food applications such as salads and other vegetable products, drinks and dairy products, ready-to-eat meals and some types of fish as for example shrimps, due to processing spreed, costs, consumer preference and influence on the texture and/or taste

Thus, said above-mentioned methods of salt addition, pH regulation, nitrite addition and processing techniques as e.g. heat treatment are not always satisfactory for the purpose of preservation of foods, especially when controlling gram-negative bacterial pathogens. Accordingly, the preservation of protein-containing food products, pH sensitive food products and refrigerated food products such dairy products, salads and other vegetable products, dried foods and convenient foods as e.g. ready-to-eat meals, and especially meat (=including fish and poultry) products still proves to be a problem, especially if the food product needs to be protected against food poisoning as consequences of e.g. temperature-abuse and/or contamination of food. It is known that one of the most important causes of food poisoning is contamination due to incorrect handling of food products. Furthermore, products are often stored at improper conditions. Temperature-abuse (e.g. incidental storage at high temperature) can cause the in the product already present but inactivated bacteria to grow again resulting in food poisoning by pathogenic bacteria. The invention provides an effective alternative to overcome the above-mentioned problems in preservation of foods against food poisoning and further provides a means for fighting food poisoning by pathogenic bacteria of food and products due to e.g. temperature-abuse and/or contamination due to e.g. improper handling and/or improper preparation. It is known that combinations of containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids and some natural products like phytotherapeutic plant extracts can be used to prevent growth of bacteria which cause food spoilage, also called putrefaction. Normally these are lactic acid bacteria, i.e. gram-positive bacteria. When food is spoiled, the taste and/or its appearance is affected, but the consumers health is not at stake. The present invention, however, is directed to the prevention of food poisoning. Food poisoning is caused by gram-negative bacterial pathogens and fungie such as as Fusarium spp., e.g. Fusarium semitectum, Fusarium moniliforme, Fusarium solani, Fusarium oxysporum; Verticillium spp., e.g. Verticillium theobromae; Nigrospora spp.; Botrytis spp., e.g. Botrytis cinerea; Geotrichum spp., e.g. Geotrichum candidum; Phomopsis spp., Phomopsis natalensis; Diplodia spp., e.g. Diplodia citri; Alternaria spp., e.g. Alternaria citri, Alternaria alternata; Phytophthora spp., e.g. Phytophthora citrophthora, Phytophthora fragariae, Phytophthora cactorum, Phytophthora parasitica; Septoria spp., e.g. Septoria depressa; Mucor spp., e.g. Mucor piriformis; Monilinia spp., e.g. Monilinia fructigena, Monilinia laxa; Venturia spp., e.g. Venturia inaequalis, Venturia pyrina; Rhizopus spp., e.g. Rhizopus stolonifer, Rhizopus oryzae; Glomerella spp., e.g. Glomerella cingulata; Sclerotinia spp., e.g. Sclerotinia fruiticola; Ceratocystis spp., e.g. Ceratocystis paradoxa; Penicillium spp., e.g. Penicillium funiculosum, Penicillium expansum, Penicillium digitatum, Penicillium italicum; Gloeosporium spp., e.g. Gloeosporium album, Gloeosporium perennans, Gloeosporium fructi genum, Gloeosporium singulata; Phlyctaena spp., e.g. Phlyctaena vagabunda; Cylindrocarpon spp., e.g. Cylin drocarpon mali; Stemphyllium spp., e.g. Stemphyllium vesicarium; Phacydiopycnis spp., e.g. Phacydiopycnis malirum; Thielaviopsis spp., e.g. Thielaviopsis paradoxy; Aspergillus spp., e.g. Aspergillus niger, Aspergillus carbonarius; Nectria spp., e.g. Nectria galligena; Pezicula spp., Escherichia Coli, Enterobacter sakazakii, Salmonella, pseudomonas, escheria coli, entereobacter aerogenes, coliform, legionalla, Salmomella spp., Campylobacter spp., Staphylococcus aureus, Listeria monocytogenes, Bacillus cereus, Clostridium spp., Escherichia coli O157:H7, Shigella spp., Yersinia enterocolitica, Vibrio spp., Brucella spp. ve Aeromonas spp. and Campylobacter said pathogens produce toxine and/or cause infections.

1. The use of a polymeric guanidine derivative based on a diamine containing oxyalkylene chains between two amino groups, with combination polymeric guanidine derivative based on a diamine containing oxyalkylene chains between two amino groups, with the guanidine derivative representing a product of polycondensation between a guanidine acid addition salt and a diamine containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids and some natural products like phytotherapeutic plant extracts

2. The use according to claim 1, characterized in that, among the representatives of the family of polyoxyalkylene guanidine salts, there are such using triethylene glycol diamine (relative molecular mass: 148), polyoxypropylene diamine (relative molecular mass: 230) as well as polyoxyethylene diamine (relative molecular mass: 600).

3. The use according to any of claim 1 or 2, characterized in that poly-[2-(2-_ethoxyethoxyethyl)_guanidinium hydrochloride] having at least 3 guanidinium groups is used.

4. The use according to claim 3, characterized in that the average molecular mass of the drug substance ranges from 500 to 3,000.

5. The use according to any of claims 1 to 4, characterized in that the drug composition is designed as a drug composition for veterinary use.

6. The use according to any of claim 1, characterized in that, combination guanidinium derivatives, particularly to combination polymeric guanidine derivative based on a diamine containing oxyalkylene chains between two amino groups, with the guanidine derivative representing a product of polycondensation between a guanidine acid addition salt and a diamine containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids and some natural products like phytotherapeutic plant extracts can apply at least 3000 mg/l, preferably at least 2000 mg/l, more preferably at least 2000 mg/l to protecting agent from pathogens organism at surface, air, textile, paint, plastic, silicone and wood, polyethylene and derivatives

7. The composition according to claim 1, wherein the guaninidium compound is oligo(2-(2-ethoxy)ethoxy ethyl guanidinium chloride).

8. The composition according to claim 1, where in the guaninidium compound is poly(hexamethylendiamine guanidiniumchloride), and pH of synthesis of product which combination polymeric guanidine derivative based on a diamine containing oxyalkylene chains between two amino groups, with the guanidine derivative representing a product of polycondensation between a guanidine acid addition salt and a diamine containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids and some natural products like phytotherapeutic plant extracts protecting composition according to claim 1 characterised in that it has a from 5.5 to 7.5.

9. The use according to claim 1, characterized in that, the synthesis product based on a diamine containing oxyalkylene chains between two amino groups, with the guanidine derivative representing a product of polycondensation between a guanidine acid addition salt and a diamine containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids and some natural products like phytotherapeutic plant extracts protect surface, air, textile, paint, plastic, silicone and wood, polyethylene and derivatives which apllied the general structure of textile, paint, plastic, silicone and wood, polyethylene materials.

10. The method of claim 1, where in herbal plants are Aniseed (Anisi fructus), Barbados Aloes (Aloe barbadensis), Bearberry leaf (Uvae ursi folium), Bilberry Fruit (Myrtilli fructus), Birch Leaf (Betulae folium), Black Cohosh (Cimicifugae rhizoma), Black Currant Leaf (Ribis nigri folium), Black Horehound (Ballotae nigrae herba), Bogbean leaf (Menyanthidis trifoliatae folium), Burdock Root (Arctii radix), Butcher's Broom (Rusci rhizome), Cape Aloes (Aloe capensis), Cascara (Rhamni purshianae cortex), Centaury (Centaurii herba), Clove oil (Caryophylli aetheroleum), Cola (Colae semen), Comfrey root (Symphyti radix), Couch Grass Rhizome (Graminis rhizoma), Elder flower (Sambuci flos), Feverfew (Tanaceti parthenii herba), Frangula Bark (Frangulae cortex), Gentian Root (Gentianae radix), Grindelia (Grindeliae herba), Hamamelis bark (Hamamelidis cortex), Hamamelis leaf (Hamamelidis folium), Hamamelis water (Hamamelidis aqua), Hydrastis rhizoma (Goldenseal rhizome), Ispaghula Husk (Plantaginis ovatae testa), Java Tea (Orthosiphonis folium), Lady's Mantle (Alchemillae herba), Linseed (Lini semen), Mallow Flower (Malvae flus), Meadowsweet (Filipendulae ulmariae herba), Melissa leaf (Melissae folium), Myrrh (Myrrha), Mullein flower (Verbasci flus), Nettle Root (Urticae radix), Pelargonium Root (Pelargonii radix), Psyllium Seed (Psylli semen), Restharrow Root (Ononidis radix), Rhatany Root (Ratanhiae radix), Ribwort Plantain leaf/herb (Plantaginis lanceolatae folium/herba), Sage Leaf, Trilobed (Salviae trilobae folium), Tormentil (Tormentillae rhizoma), White Horehound (Marrubii herbal), Wild Pansy (Violae herba cum flore), Wild Thyme (Serpylli herba), Willow Bark (Salicis cortex).

11. The use according to claim 1, characterized in that, some of the diseases which Fusarium spp., e.g. Fusarium semitectum, Fusarium moniliforme, Fusarium solani, Fusarium oxysporum; Verticillium spp., e.g. Verticillium theobromae; Nigrospora spp.; Botrytis spp., e.g. Botrytis cinerea; Geotrichum spp., e.g. Geotrichum candidum; Phomopsis spp., Phomopsis natalensis; Diplodia spp., e.g. Diplodia citri; Alternaria spp., e.g. Alternaria citri, Alternaria alternate; Phytophthora spp., e.g. Phytophthora citrophthora, Phytophthora fragariae, Phytophthora cactorum, Phytophthora parasitica; Septoria spp., e.g. Septoria depressa; Mucor spp., e.g. Mucor piriformis; Monilinia spp., e.g. Monilinia fructigena, Monilinia laxa; Venturia spp., e.g. Venturia inaequalis, Venturia pyrina; Rhizopus spp., e.g. Rhizopus stolonifer, Rhizopus oryzae; Glomerella spp., e.g. Glomerella cingulata; Sclerotinia spp., e.g. Sclerotinia fruiticola; Ceratocystis spp., e.g. Ceratocystis paradoxa; Penicillium spp., e.g. Penicillium funiculosum, Penicillium expansum, Penicillium digitatum, Penicillium italicum; Gloeosporium spp., e.g. Gloeosporium album, Gloeosporium perennans, Gloeosporium fructi genum, Gloeosporium singulata; Phlyctaena spp., e.g. Phlyctaena vagabunda; Cylindrocarpon spp., e.g. Cylin drocarpon mali; Stemphyllium spp., e.g. Stemphyllium vesicarium; Phacydiopycnis spp., e.g. Phacydiopycnis malirum; Thielaviopsis spp., e.g. Thielaviopsis paradoxy; Aspergillus spp., e.g. Aspergillus niger, Aspergillus carbonarius; Nectria spp., e.g. Nectria galligena; Pezicula spp., Escherichia Coli, Enterobacter sakazakii, Salmonella, pseudomonas, escheria coli, entereobacter aerogenes, coliform, legionalla, Salmomella spp., Campylobacter spp., Staphylococcus aureus, Listeria monocytogenes, Bacillus cereus, Clostridium spp., Escherichia coli O157:H7, Shigella spp., Yersinia enterocolitica, Vibrio spp., Brucella spp. ve Aeromonas spp. and Campylobacter was destroyed using syntheis product which containing oxyalkylene chains between two amino groups, with the guanidine derivative representing a product of polycondensation between a guanidine acid addition salt and a diamine containing polyoxyalkylene chains between two amino groups, Hexamethylenediamine (1,6-hexanediamine) guanidinium derivatives, particularly to combinations of oligo(2-(2-ethoxy)ethoxy ethyl guanidinium chloride), modified polyhexamethylene guanidine (PHMG) as an antimicrobial agent, poly(hexamethylendiamine guanidinium chloride), Polietheramine derivatives (JEFFAMINE EDR-148), Polietheramine (Triethyleneglycol diamine (TEGDA) enzymes, PGPR, amino acids, antioxidants like humic acids and some natural products like phytotherapeutic plant extracts.
